(19) United States
(12) Patent Application Publication
(10) Pub. No.: US 2025/0207156 A1
(43) Pub. Date: Jun. 26, 2025

(54) ANAEROBIC MICROORGANISM METABOLISM FOR CAPTURE OF DILUTE CARBON DIOXIDE AND CONVERSION TO REDUCED, INCLUDING SELF-SEPARATING, CARBON COMPOUNDS

(71) Applicant: The Board of Trustees of the Leland Stanford Junior University, Stanford, CA (US)

(72) Inventors: Grace Elizabeth Callander, San Francisco, CA (US); Joerg Stefan Deutzmann, Menlo Park, CA (US); Alfred Michael Spormann, Stanford, CA (US)

(21) Appl. No.: 18/990,766

(22) Filed: Dec. 20, 2024

Related U.S. Application Data
(60) Provisional application No. 63/614,348, filed on Dec. 22, 2023.

(51) Int. Cl. C12P 5/02; C02F 3/34; C02F 101/10; C02F 103/08; C12M 1/107; C12N 1/20; C12R 1/01

(52) U.S. Cl. CPC C12P 5/023; C02F 3/34; C12M 21/04; C12N 1/205; C02F 2101/10; C02F 2103/08; C12R 2001/01

(57) ABSTRACT

Integrated systems and methods are provided for non-photosynthetic microbial conversion of low concentrations of dissolved inorganic carbon (DIC), including from alkaline solutions, to capture, concentrate, and store CO₂ as methane (CH₄) biogas or any other reduced organic compound. The methods allow CO₂ capture and conversion, and can provide a source of hydrocarbons for synthesis, energy generation, carbon storage, and the like. The methods disclosed herein produce end products at high selectivity relative to chemical catalysis systems.

### BACKGROUND

By the end of this decade, gigatons of anthropogenically emitted CO₂ need to be removed from the atmosphere annually to limit global warming and ocean acidification. Removal of CO₂ from the atmosphere is thermodynamically expensive because the gas is dilute in air at 420 ppm, which corresponds to a partial pressure (pCO₂) of 42 Pa at standard atmospheric pressure.

However, CO₂ is highly soluble in aqueous solution, and when CO₂ dissolves, it forms carbonic acid, which dissociates to protons and bicarbonate (pKa 6.32) or carbonate (pKa 10.28), which are collectively (CO₂ (aq), H₂CO₃, HCO₃, CO₃²⁻) referred to as Dissolved Inorganic Carbon (DIC). The absolute amount of DIC and the relative amounts of each species are governed by the pH of the aqueous solution, along with temperature, salinity, and metal cation concentration.

As a result of this physical-chemical equilibrium, DIC concentration (cDIC) in slightly alkaline aqueous solution, including of seawater, (pH 8.1) is ˜50× higher than the average CO₂ concentration in air. In fact, an increase in aqueous solution pH from 7 to 10 with 42 Pa CO₂ in the gas phase increases cDIC from about 100 μM to 100 mM, where the majority of DIC is in the bicarbonate form. Capture of atmospheric CO₂ in alkaline solution to form DIC has received considerable research attention, but further processing of the DIC is required to keep the carbon sequestered over long time periods. The disclosure herein addresses this need.

### SUMMARY

Integrated systems and methods are provided for non-photosynthetic microbial conversion of dissolved inorganic carbon (DIC) from alkaline solutions to capture, concentrate, and store CO₂ from dilute sources as methane (CH₄) biogas. The methods allow carbon capture, and can provide a source of hydrocarbons for synthesis, energy generation, and the like. The methods disclosed herein produce end products at high selectivity relative to chemical catalysis systems. The process is tolerant to components of biogas products, e.g. hydrogen sulfide, sulfur oxides, and nitrous oxides, etc.

It is shown herein that anaerobic methanogens, e.g. alkalitolerant hydrogenotrophic methanogens such as members of the Methanopyrales, Methanococcales, and Methanobacteriales, as well as Methanomicrobiales, Methanocellales, and Methanosarcinales can metabolize dissolved inorganic carbon (DIC), e.g. in the form of bicarbonate in sea water, at low, including atmospheric, concentration, and convert to more reduced compounds such as methane, which can be readily separated from a microbial reactor. In some embodiments the methanogen is Methanococcus vannielii. This process can utilize the effect of alkaline aqueous equilibrium of carbon dioxide to naturally enrich carbon dioxide from air as dissolved inorganic carbon (DIC) in aqueous solution.

In some embodiments a method is provided for the microbial conversion of dissolved inorganic carbon (DIC) in an alkaline aqueous solution to methane, the method comprising contacting the alkaline solution with an effective dose of an alkalotolerant hydrogenotrophic methanogen; and maintaining for a period of time sufficient to convert the DIC to methane. In some embodiments the alkaline solution is a minimal aqueous medium containing all necessary mineral salts, nutrients, and trace elements. In some embodiments the alkaline solution is seawater, optionally treated to increase alkalinity. In some embodiments the methane is removed from the system, and optionally converted to a compound such as longer chain hydrocarbons or carbon fibers or carbon nanotubes. In some embodiments the methane is utilized for energy, electricity production, etc. The methods can be performed in moderately alkaline solution with mineral salt inputs at ambient temperatures and pressures, for example at 1 bar from about 0° to about 30° C. In some embodiments, the oxygen removal or conversion process takes place at elevated temperatures up to 80° C.

The pH of the solution may be from around pH 7 to about pH 10.5, e.g. about pH 7.5, about pH 8, about pH 8.5, about pH 9, about pH 9.5, about pH 10, about pH 10.5 or more. In some embodiments the PH is from about pH 7.5 to about pH 10, from about pH 8 to about pH 9, from about pH 8 to about pH 8.5. The DIC increases with pH, and a pH level may be selected that balances the cost of increasing alkalinity with the benefit of increasing levels of DIC.

The concentration of DIC can vary with pH, and may be from about 100 μM to 100 mM, e.g. from about 1 mM to about 100 mM, from about 1 mM to about 50 mM, from about 1 mM to about 25 mM, from about 0.5 mM to about 10 mM.

The method is usually performed in anoxic conditions, e.g. where the level of O₂ in head space is less than about 5 mg O₂ L⁻¹, less than about 1 mg O₂ L⁻¹, less than about 500 μg O₂ L⁻¹, less than about 100 μg O₂ L⁻¹, less than about 50 μg O₂ L⁻¹, less than about 10 μg O₂ L⁻¹. The level of dissolved O₂ in the solution may be around or less than 500 μM O₂. In some embodiments the method is performed in a contained system, such as a bioreactor, that allows such anaerobic conditions. In some embodiments, O₂ present from environmental sources is removed from a solution containing DIC using chemical removal or membrane-based separations. In some embodiments oxygen-consuming microbial strains or communities are utilized to remove such residual O₂.

The process may be done at any suitable temperature. For example, the temperature may be about 5° C., about 10° C., about 15° C., about 20° C., about 25° C., about 40° C. In an embodiment, the temperature is from about 10 to 90° C.

In some embodiments a system is provided for CO₂ conversion by non-photosynthetic microbial conversion of dissolved inorganic carbon (DIC) from alkaline solutions. The system comprises: a microbial bioreactor comprising an effective dose of an alkalotolerant hydrogenotrophic methanogen, and an input of an alkaline solution as described above, comprising dissolved inorganic carbon (DIC). The bioreactor may comprise an input or component for removal of excess O2. The bioreactor may comprise an input for a suitable alkaline solution, e.g. seawater optionally treated to increase alkalinity.

In some embodiments, there is a separate reactor for alkaline CO₂ capture. This reactor may be a high flux system such as a trickle bed reactor or an ambient process such as a high surface area reservoir exposed to the gas stream. The solution supplied for alkaline capture may be a fresh aqueous stream or the recirculated effluent after microbial CO₂ reduction.

In some embodiments the solution in the bioreactor is exchanged periodically. In some embodiments the bioreactor is supplied with a continuous inflow of solution. In some embodiments the electron donor for CO₂ reduction is hydrogen. In some embodiments the bioreactor medium is maintained without exchange for an extended period of time, e.g. for up to about 10 days, for up to about 20 days, for up to about 30 days, for up to about 40 days, for up to about 50 days, for up to about 75 days, for up to about 100 days or more. In some embodiments the solution is flowed over a support material in the reactor such that a biofilm forms. The substrates may be delivered co-currently or countercurrently with different or equal flow rates.

The system may comprise a second element comprising a methane storage unit operably connected to the bioreactor and capable of storing methane produced by the bioreactor. The storage unit may comprise compressors, monitoring devices, out and input regulators and the like. A number of configurations can be used for the methane storage unit, which can be configured in a unit with the bioreactor, or may be remote, e.g. converted to liquid natural gas or a natural gas grid. For example, the storage unit may use a conventional storage configuration, where methane is stored as compressed natural gas (CNG) at 207 bar in pressure vessels utilizing a compression stage. As an alternative, adsorbed natural gas is stored as an adsorbed phase in a porous solid at a lower pressure, e.g. at greater than about 50 bar, greater than about 100 bar, greater than about 150 bar as 150 v/v. Adsorbents with high methane capacity include, for example, activated carbons; zeolites, metal-organic frameworks, and crystalline materials formed from metal-oxide clusters bridged by functionalized organic links and known as isoreticular metal-organic frameworks (IRMOFs).

An optional third element may be operably connected to the methane storage unit, which third element converts methane to electricity or other forms of energy, such as thermal, or a combination thereof, which third element may be linked to a transformer or other unit for transferring electric energy in a useful format. Another element that may be operably connected to the methane production, or the methane storage unit is an element that converts methane to inert carbon compounds such as carbon nanotubes, graphitic carbon, amorphous, or other climate-inert carbon.

The system can be scaled as appropriate for the needs of the user, where, for example, a bioreactor can be operated at up to about 10 liter volume, up to about 50 liter volume, up to about 100 liter volume, up to about 500 liter volume, up to about 1000 liter volume, up to about 10,000 liter volume, or more. The storage and combustion elements can be appropriately sized for the bioreactor.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. DIC concentration and relative DIC species within physiological growth pH range of M. vannielii. Values in plot are determined from CO₂SYS model, using constants (Keq) in pure water at 25° C. and 1 atm (Pierrot et al. 2012).

FIG. 2. Growth curves of M. vannielii in DIC buffered medium with varied starting pH measured over several days. pH (A), log 10 of pCO₂ in headspace (B), Optical density (C), methane production (D) measured over time in serum bottles at 30° C. Note: Though measurement of pHₜ₌₀ 9.8 bottles continued for 500 hr, x-axis truncated to 100 hr in A and B and 150 hr in C and D. There was no noticeable change in growth behavior after 100 hr.

FIG. 3. Specific methane formation rates in (A) pH 7, (B) pH 8, and (C) pH 9 medium with 0-10 mM DIC or 80-100 mM DIC. Rate data fit with Monod equation using apparent K_{M} for cDIC and vₘₐₓ values from Lineweaver Burk plots (values in Table 1). Values are indicated with points and dashed line shows 95% confidence interval.

FIG. 4. Reduction of CO₂ from room air to CH₄ with with chemical and biological O₂ removal and biological CH₄ formation. Seven bottles were prepared with air-saturated alkaline medium and N₂ (bottle 1) or H₂ (bottle 2-7) in the headspace. Biological oxygen scavengers, C. necator, were added to bottles 1, 3, 5, and 6 at t=0 hours. A chemical oxygen reducing compound, Na₂S, was added to bottles 4 and 7 at t=0 hours. M. vannielii was added to bottles 5-7 at t=120 hours. At t=190 hours, inoculum from bottles 5, 6, and 7 was transferred to bottles 2, 3, and 4, respectively. CH₄ production was measured with gas chromatography and pressure gauges (A, B). CO₂ consumption was measured relative to a 420 ppm CO₂ in N₂ gas standard (C). Cell density in each bottle was measured as optical density at 600 nm (D). pH of the medium was measured with a Thermofisher pH probe (E). Only bottles where oxygen had been sufficiently removed and H₂ was available for CO₂ reduction produced CH₄ (bottles 3, 4, 5, 6, and 7).

FIG. 5. Reduction of 5% CO₂-enriched air to bio-CH₄ with a microbial co-culture. Alkaline medium with 5% CO₂-enriched air in the headspace (black) was fed to a trickle bed reactor consisting of two columns. pCO₂, pH₂, and pCH₄ (A, C, D) were measured in the O₂ removal column (green) then the anaerobic CO₂ reduction column (red). DIC was measured in the medium and outflow from each of the columns (B). Continuous CH₄ production was demonstrated from 5% CO₂-enriched air for 18 days. C. n.: Cupriavidus necator, M. v.: Methanococcus vannielii

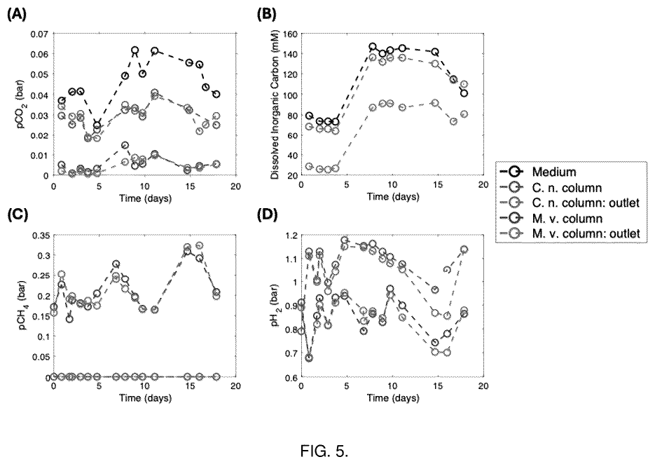

FIG. 6. pCO₂ to DIC calibration curve from integrated gas chromatography peaks in MOPS buffered pH 7 mineral medium.

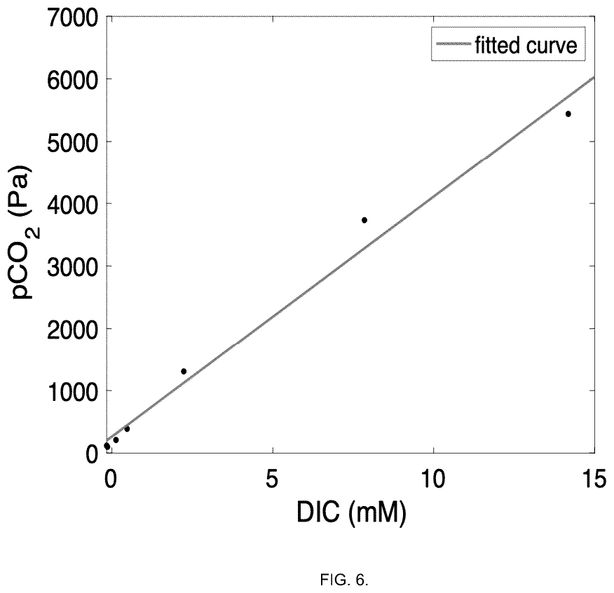

FIG. 7. Optical Density, pH, Total pressure, Methane production, and CO₂ consumption measured for buffered experiments with 0-10 mM added DIC at pH 7 (A-E), pH 8 (F-J), and pH 9 (K-O). Purple points indicate measurements from serum bottles with cells, while black points indicate measurements from serum bottles with sterile-filtered inoculum.

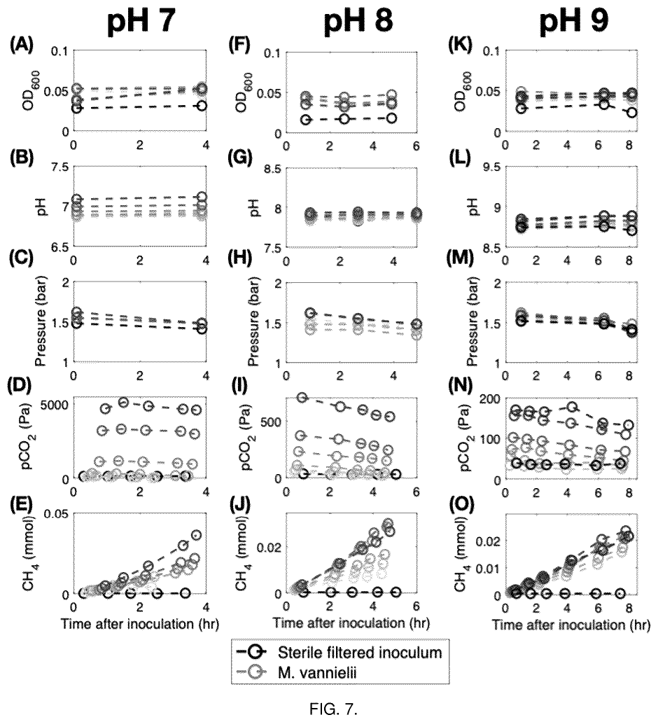

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Comparable cell” shall mean a cell whose type is identical to that of another cell to which it is compared. Examples of comparable cells are cells from the same microbial strain.

“Suitable conditions” shall have a meaning dependent on the context in which this term is used. For example, when used in connection with contacting an agent to a cell, this term shall mean conditions that permit an agent capable of doing so to enter a cell and perform its intended function. In one embodiment, the term “suitable conditions” as used herein means physiological conditions. Suitable conditions may also include conditions where the fed substrate, such as DIC or CO₂, is reduced by the organism or enzymes, without optimal cell viability.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

Anoxic and Anaerobic. As used herein the term “anoxic” refers to conditions of low molecular oxygen, for example where the level of O₂ is less than about 5 mg O₂ L⁻¹ of head space, less than about 1 mg O₂ L⁻¹, less than about 500 μg O₂ L⁻¹, less than about 100 μg O₂ L⁻¹, less than about 50 μg O₂ L⁻¹, less than about 10 μg O₂ L⁻¹. The level of dissolved O₂ in the solution may be less than about 1000 μM O₂, less than about 750 μM O₂, less than about 500 μM O₂. Anaerobic organisms grow under anoxic conditions, and may be facultative or obligate anaerobes.

Alkalotolerant hydrogenotrophic methanogens belong to the domain Archaea, specifically within the phylum Euryarchaeota. This methanogenic archaeon is characterized by its ability to thrive in environments with elevated pH levels, showcasing a remarkable tolerance to alkaline conditions. Unlike many other microorganisms that are sensitive to changes in pH, alkalotolerant hydrogenotrophic methanogens have adapted mechanisms to maintain their metabolic activities and methane production in alkaline settings.

Taxonomically, these methanogens may be found in the orders Methanobacteriales, Methanococcales, Methanomicrobiales, Methanopyrales, Methanocellales and Methanosarcinales, and families Methanococcus and Methanomicrobiaceae, where specific genera include representatives of alkalotolerant hydrogenotrophic methanogens. These microorganisms play a crucial role in anaerobic environments, contributing to the carbon cycle by producing methane through the reduction of carbon dioxide using molecular hydrogen. Their ability to function in alkaline conditions distinguishes them from other methanogens and highlights their ecological significance in environments with elevated pH, such as alkaline sediments and certain industrial processes.

Within the family of Methanococcus, Methanococcus aeolicus, Methanococcus maripaludis, Methanococcus vannielii, Methanococcus voltae are of interest, particularly Methanococcus vannielii. Within the family of Methanomicrobiaceae, the genus Methanoculleus, e.g. the species Methanoculleus bourgensis, Methanoculleus chikugoensis, Methanoculleus horonobensis, Methanoculleus hydrogenitrophicus, Methanoculleus marisnigri, Methanoculleus palmolei, Methanoculleus receptaculi, Methanoculleus sediminis, Methanoculleus submarinus, Methanoculleus taiwanensis, Methanoculleus thermophilus, etc.; the genus Methanofollis, e.g. the species Methanofollis aquaemaris, Methanofollis ethanolicus, Methanofollis fontis, Methanofollis formosanus, Methanofollis liminatans, Methanofollis propanolicus, Methanofollis tationis, etc. the genus Methanogenium, e.g. the species Methanogenium boonei, Methanogenium cariaci, Methanogenium frigidum, Methanogenium marinum, Methanogenium organophilum, etc., the genus Methanolacinia, e.g. the species Methanolacinia paynteri, Methanolacinia petrolearia, etc., the genus Methanomicrobium, e.g. the species Methanomicrobium antiquum, Methanomicrobium mobile, etc., the genus Methanoplanus, e.g. Methanoplanus endosymbiosis, Methanoplanus limicola, etc., the genus Methanovulcanius, e.g. Methanovulcanius yangii, the genus Methanocalculus, e.g. Methanocalculus natronophilus, Methanocalculus alkaliphilus, may find use in the methods of the disclosure. Other strains that are alkalotolerant or alkalophilic include Methanosalsum zhilinae (Kendall and Boone, 2006) and Methanothermococcus thermolitotrophicus.

The methanogenic strain can also be added as part of a defined or undefined microbial community. In this instance, the other members of the microbial community can perform auxiliary functions, such as supplying vitamins or reduced sulfur compounds (such as H₂S), reducing effective oxygen concentration, or other functions.

An effective dose of the alkalotolerant hydrogenotrophic methanogens for conversion of DIC to methane may be at least an initial concentration of at least about 10 cells/liter, at least 10² cells/liter, at least 10³ cells/liter, at least 10⁴ cells/liter, at least 10⁶ cells/liter, at least 10⁶ cells/liter, or more. The methanogen may be initially provided as a concentrated inoculum, e.g. an inoculum of at least 10⁷ cells/liter, at least 10⁸ cells/liter, at least 10⁹ cells/liter, at least 10¹⁰ cells/liter, at least 10¹¹ cells/liter, at least 10¹² cells/liter, or more. A culture may be initiated under controlled condition, e.g. in a laboratory setting.

Anaerobic methanogenesis is the process by which microorganisms, e.g. alkalotolerant hydrogenotrophic methanogens as disclosed herein, reduce dissolved CO₂ or any other form of DIC to methane. As disclosed herein, this process or other catabolic CO₂ reduction pathways, combined with the increased concentration of DIC in alkaline aqueous solutions, can be used to reduce the DIC in equilibrium with atmospheric CO₂ and to generate hydrocarbons and other reduced carbon compounds. Due to the anoxic requirements of the enzymes and microbes, O₂ is excluded from the reactions by physical containment, chemical reactions, or biological means.

Anaerobic methanogenesis can be designed and engineered to operate using a number of different configurations and can be categorized into batch vs. continuous process mode, mesophilic vs. thermophilic temperature conditions, single stage vs. multistage, etc. processes.

Anaerobic methanogenesis can be performed as a batch process or a continuous process. In a batch system, the alkaline solution is added to the reactor at the start of the process. The reactor is then sealed for the duration of the process. In continuous digestion processes, the alkaline solution is constantly added or added in stages to the reactor. End products are constantly or periodically removed or self-separated, resulting in constant production of methane or other reduced carbon compounds. The residence time in the bioreactor varies with the amount and type of feed, and with the configuration of the system, and may vary from days to weeks to several months.

Self-separating compounds, for the purposes of the disclosure, refers to compounds such as methane that have a sufficiently low solubility in the alkaline solution such that the compounds as produced during the process of the disclosure will partition away from the reaction solution.

A microbial bioreactor is a specialized device designed for the cultivation and manipulation of microorganisms in a controlled environment, particularly an anaerobic environment. The microbial bioreactor typically consists of a vessel optionally equipped with sensors and control systems to monitor and regulate critical parameters, including temperature, pH, dissolved oxygen, and nutrient concentrations. The vessel is often made of materials that are biocompatible and resistant to corrosion, and it may have features such as impellers or stirrers to enhance mixing and oxygen transfer within the culture. Microorganisms are introduced into the bioreactor along with a suitable medium.

Various salts and buffers may be included, for example to increase alkalinity of the solution, e.g. Mg(OH)₂, Ca(OH)₂, such wollastonite, olivine, and anorthite; NaOH, KOH, carbonate, such as calcite, dolomite, quicklime (calcium oxide, CaO), portlandite (calcium hydroxide, Ca(OH)₂); etc. As discussed herein, raising the pH increases the concentration of DIC under normal atmospheric conditions. Suitible buffers include Borates (HBO₄) or Good's buffers, such as EPPS, HEPBS, MOPS, Tris, CHES, CAPSO, CABS. When changing the concentration of a particular component of the solution, that of another component may be changed accordingly. Also, the concentration levels of components in the reactor may be varied over time.

For example, a solution of interest, including without limitation seawater, can be made more alkaline by increasing mineral dissolution, e.g. by increasing the reactive surface area of the minerals through pulverizing alkaline rock into small particles. Methods of enhancing alkalinity in seawater include the dissolution of naturally occurring silicate-based minerals such as olivine, accelerated limestone weathering, and dissolution of calcium carbonate derivatives such as quicklime (calcium oxide, CaO) or portlandite (calcium hydroxide, Ca(OH)₂) in the surface ocean. The impact of mineral addition is specific to the mineral itself and technology. Seawater can also be made more alkaline by electrochemical electrolysis, e. g., by producing hydroxide on an electrode.

In a semi-continuous operation mode, the reactor may be operated in dialysis, diafiltration batch or fed-batch mode. A feed solution may be supplied to the reactor through the same membrane or a separate injection unit. A carbon neutral or negative commodity chemical of interest may be removed from the recycled stream. The reactor may be stirred internally or by proper agitation means, including by gas sparging or by directing the flow of a recycle inlet stream. The amount of methane produced can be determined using any instrument that measures the concentration in the stream. The product may be separated with or without temperature, pressure, or pH swings.

In some embodiments a bioreactor is an anaerobic Membrane Bioreactor (AnMBR). AnMBR provides a membrane-coupled system to facilitate independent control of the hydraulic, gas, and solid retention within the reactor. An AnMBR can facilitate product removal from the microbial broth. AnMBR can also facilitate retention of microorganisms. An anaerobic membrane bioreactor system can include a cross-flow membrane unit for the solid-liquid separation of the bioreactor effluent. The cross-flow membrane unit may be a cross-flow microfiltration or ultrafiltration unit. A portion of the retentate stream from the membrane separation unit with relatively higher concentration of anaerobic microorganisms is recycled back to the anaerobic bioreactor, thus making it possible to carry out the operation at higher biomass concentrations. This higher concentration of anaerobic microorganisms in the bioreactor reduces the hydraulic retention time, and enhances efficiency. A portion of the retentate stream from the membrane separation unit can be sprayed from the operative top of the bioreactor in the headspace to contact the stream with the DIC or CO₂ to facilitate the conversion process. The continuous recycling of the retentate stream in the bioreactor can increase the consumption of carbon dioxide as DIC in the solution.

In some embodiments a bioreactor is a trickle bed reactor, which provides a hydrophilic support material for the immobilization of microorganisms or enzymes. A biofilm may develop in the reactor, allowing for higher gas flow rate than liquid flow rates.

In some embodiments a bioreactor is a bioelectrochemical reactor, which provides a membrane-separated cathode compartment to provide reducing equivalents to the cells. Enzymes immobilized on the electrode or in the solution may facilitate electron transfer for CO₂ reduction. Reducing equivalents could be any reduced molecule produced at the cathode, but mainly refers to hydrogen, carbon monoxide, or formic acid produced electrochemically on the cathode or to electrons taken up by the microbes directly from the electrode. In some embodiments, the microbes are present in the cathode compartment to facilitate the cellular uptake of reducing equivalents and reduce mass transfer limitations. This allows supply of hydrogen or other reducing equivalents to the cells with exceptional control. Bioelectrochemical reactors are comprised of an anode compartment containing an anode capable of performing an oxidation reaction, such as water oxidation to oxygen, and a cathode compartment containing a cathode capable of performing a reduction reaction, such as water reduction to hydrogen. Both compartments can be separated by a separator, such as an anion exchange membrane, a cation exchange membrane, a bipolar membrane, a porous separator, or a diffusive barrier to limit or prevent the crossover of the reduced and oxidized molecules.

### Process Methods

Methods are provided for the microbial conversion of dissolved inorganic carbon (DIC) in an alkaline solution to methane, the method comprising contacting the alkaline solution with an effective dose of an alkalotolerant hydrogenotrophic methanogen; and maintaining for a period of time sufficient to convert the DIC to methane where electrons for the reduction are delivered as hydrogen or other suitable electron donors. In some embodiments the alkaline solution is seawater, optionally treated to increase alkalinity. In some embodiments the methane is removed from the system, and optionally converted to a compound such as longer chain hydrocarbons or carbon of various morphologies (ex. graphene, amorphous, fibers or tubes). In some embodiments the methane or further modified methane is utilized as a fuel for energy, electricity production, etc. The methods for CO₂ reduction can be performed in near neutral pH solution up to very alkaline solution with mineral salt inputs at ambient temperatures and pressures, for example 1 bar at from about 0° to about 30° C. pH may range, for example, from about 6 to about 12, from about 7 to about 10, from about 7 to about 8.

The reactions may utilize a large scale reactor, small scale, or may be multiplexed to perform a plurality of reactions. Continuous reactions will use a feed mechanism to introduce a flow of solution, and may isolate the methane as part of the process. Batch systems are also of interest, where additional reagents may be introduced over time to prolong the period of time for synthesis or to limit the production of side products. A reactor may be run in any mode such as batch, extended batch, semi-batch, semi-continuous, fed-batch and continuous, and which will be selected in accordance with the application purpose.

The reactions may be of any volume, either in a small scale, usually at least about 1 ml and not more than about 15 ml, or in a scaled up reaction, where the reaction volume is at least about 15 ml, usually at least about 50 ml, more usually at least about 100 ml, and may be 500 ml, 1000 ml. The reaction volume may be greater than 1 L such as about 5 L, about 10 L, about 100 L, about 200 L, about 500 L, about 1000 L, about 5000 L, about 10000 L, about 20000 L, about 30000 L, about 40000 L, about 50000 L, about 100000 L, or greater. Reactions may be conducted at any scale. The technology is deployable at large scale, can easily be implemented by building production facilities, for example near seawater facilities.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Alkaline Hydrogenotrophic Methanogenesis in Methanococcus vannielii at Low Carbon Dioxide Concentrations

Capture of carbon dioxide from the atmosphere is challenging and thermodynamically expensive because of its dilute concentration. Utilizing the concentrating effect of dissolving atmospheric CO₂ in alkaline solution to form dissolved inorganic carbon (DIC), we explored the potential of an alkalotolerant hydrogenotrophic methanogen, here Methanococcus vannielii, to capture and convert CO₂ at low partial pressures to methane. Kinetic analysis of CO₂ reduction to CH₄ at low pCO₂ at pH 7, 8, and 9 revealed fastest rates at pH 8 with apparent Km of 135 Pa CO₂ or 1.6 mM DIC and a vmax of 9.7 mmol CH₄ L⁻¹ OD⁻¹ hr⁻¹. Using atmosphere-equilibrated DIC concentrations at pH 7, 8, and 9, methane formation rates were 1.1, 2.3, and 2.1 mmol CH₄ L⁻¹ OD⁻¹ hr⁻¹, respectively. Our data show that alkaline hydrogenotrophic methanogenesis is an alternative to photosynthetic CO₂ fixation for biological capture and conversion of CO₂ at atmospheric concentrations at reasonable rates.

Here, we show non-photosynthetic microbial conversion of DIC from alkaline solutions to capture, concentrate, and store the carbon in atmospheric CO₂ as CH₄. While plants access dilute CO₂ for carbon fixation through photosynthesis, some anaerobic microbes, such as methanogens and acetogens, utilize CO₂ as both catabolic electron acceptor and carbon source. For example, hydrogenotrophic methanogens catalyze in their energy metabolism the following reaction: 4 H₂+CO₂->CH₄+2 H₂O, ΔG*′=−131 KJ/mol, where CO₂ and not bicarbonate is the substrate of the first and only CO₂-reducing reaction (formylmethanofuran dehydrogenase). Catabolic CO₂ reduction in methanogenesis also proceeds at a high product selectivity (80-90%) compared to photosynthesis due to low ATP yield per mol CH₄. At the industrial level, hydrogenotrophic methanogenesis has been demonstrated as an important subset of Power-to-Gas technologies, where pure methanogen cultures continuously produce methane from concentrated CO₂.

Autotrophic methanogens typically exist in natural anoxic environments, such as marine or freshwater sediments or the rumen of ruminants, which contain CO₂ in form of bicarbonate at 10 s-100 s mM concentration. Autotrophic methanogens have also been isolated at elevated pH with high bicarbonate concentrations. In this work, we explore whether the alkalotolerant hydrogenotrophic Methanococcus vannielii has the potential to metabolize, under mild alkaline conditions, the naturally enriched DIC that is in equilibrium with 420 ppm gaseous CO₂. M. vannielii was isolated from San Francisco Bay mud flats and grows between pH 7 and pH 10 with only H₂ or formate as electron donor. M. vannielii is compatible with microbial electrosynthesis reactors and grows at high rates and with high product yields among characterized methanogens, likely due to its lack of cytochromes. Our study quantifies the relative rates of methane production from low pCO₂ and cDIC to explore the feasibility of alkaline methanogenesis as a dilute CO₂ conversion technology.

### Materials and Methods

Mineral medium preparation. Mineral medium was prepared with 0.34 g KCl, 2 g MgCl₂×6 H₂O, 1.5 g MgSO₄×7 H₂O, 0.25 g NH₄Cl, 0.014 g CaCl₂)×2 H₂O, 0.14 g K₂HPO₄, 18 g NaCl, 0.5 mL Na-resazurin solution (0.1% w/v), 1 mL selenite-tungstate solution, 1 mL SL-10 solution, and MilliQ H₂O to produce a total volume of 1 L. Selenite-tungstate solution contained 0.5 g NaOH, 3 mg Na₂SeO₃×5H₂O, 4 mg Na₂WO₄×2 H₂O, and MilliQ H₂O to produce a total volume of 1 L. Trace element solution SL-10 contained 10 mL HCl (25%), 1.5 g FeCl₂×4 H₂O, 70 mg ZnCl₂, 100 mg MnCl₂×4H₂O, 6 mg H₃BO₃, 190 mg CoCl₂×6 H₂O, 2 mg CuCl₂×2 H₂O, 24 mg NiCl₂×6 H₂O, 36 mg Na₂MoO₄×2 H₂O, and MilliQ H₂O to produce a total volume of 1 L. The SL-10 and SeWo solutions were filter-sterilized and stored at 4° C. (Kracke et al. 2020).

Mineral medium was boiled to remove O₂ and distributed in 25 mL aliquots to 115 mL serum bottles under 100% N₂ (4.5, Linde). N₂-gassed bottles were closed with rubber butyl stoppers, crimped, and autoclaved for 30 minutes. After autoclaving, bottles were flushed with 100% H₂ (4.5, Linde) at room temperature for 30 seconds and then filled to 1.5×10⁵ Pa. The headspace of each bottle was measured to confirm <10% N₂ concentration, <1% CH₄, and <0.04% CO₂. Medium was reduced with 1.2 mM Na₂S and 1.6 mM Cysteine-HCl from anoxic, sterile stock solutions. Anoxic buffer or DIC were added from stock solutions (described below) before each experiment.

Stock solutions for non-CO₂ based buffers were prepared from free acid salts with final concentrations of 1.5 M MOPS (pKa 7.15), 1.1 M EPPS (pKa 8.00), and 900 mM Borate (pKa 9.14). The pH of each stock solution was adjusted with NaOH pellets and measured with a double junction pH probe (Orion 9110DJWP, Thermofisher) at room temperature. Stock solutions were boiled, flushed with N₂, autoclaved, and stored at room temperature. MOPS and EPPS were selected specifically for their lack of amino groups to avoid carbamate formation with CO₂. The effect of each buffer on growth was tested in parallel triplicate batch cultures from the same preculture. OD and pH were measured over time to confirm pH was stable and the buffer did not inhibit growth.

DIC stock solutions were prepared with sodium carbonate and sodium bicarbonate, then adjusted to the appropriate pHs of 7.0, 8.0, 9.0 with 5 M NaOH or 3% HCl at room temperature. The solutions were boiled, then transferred to serum bottles that had been flushed with N₂. Each bottle's headspace was again quickly exchanged with N₂, then the bottle was sealed and autoclaved. The concentration and pH of all DIC stock solutions were verified with DIC acid tests and pH probe measurements, respectively.

DIC acid tests measure samples' cDIC by acidifying known volumes of solution in a closed vial such that all bicarbonate and carbonate are converted into carbonic acid or CO₂. To prepare each DIC acid test vial, 10 mL Agilent glass vials were flushed with N₂. While flushing, 0.85 mL 800 mM HCl was added. The vials were sealed and stored at room temperature. To measure cDIC, 0.1-0.5 mL sample was added to a vial through a syringe, vortexed, and the headspace CO₂ concentration was determined after equilibration with gas chromatography. The uncertainty of DIC acid tests was determined to be less than 5% by repeating tests six times with the same DIC stock.

Pure culture growth and sampling protocols. Methanococcus vannielii SB (DSM 1224) was purchased from the Deutsche Sammlung von Mikroorganismen und Zellkulturen GmbH (DSMZ). The strain was stored at −80° C. in anoxic glycerol stocks prepared with 0.6 mL 100% glycerol and 1.4 mL exponentially growing culture. For all experiments described here, M. vannielii was first grown in mineral medium from a glycerol stock. Then, 3.5% v/v exponentially growing inoculum was transferred to fresh medium with DIC or non-CO₂-based buffer several times to ensure acclimatization. Cupriavidus necator H16 was grown from aerobic glycerol stock in mineral medium with 140 mM glycerol and 40 mM fructose. C. necator was transferred into fresh medium with H₂, CO₂, and O₂ in the headspace to acclimatize to an autotrophic hydrogen-oxydizing growth phenotype. All cultures were handled aseptically with flame sterilization techniques and incubated on a shaker at 250 rpm in a temperature-controlled room at 30° C. Phase contrast microscopy (40×, Leica DME) was periodically used to check for contamination. Optical Density (OD) was measured at 600 nm by a UV/vis spectrophotometer (Ultraspec2100 pro, Amersham Biosciences) (Deutzmann et al. 2022). The headspace of serum bottles was sampled manually with 0.3 mL gaseous samples using a 0.5 mL glass syringe (Pressure-Lok, Vici Precision Sampling). CO₂, N₂, and CH₄ were quantified via gas chromatography (GC) on a thermal conductivity detector with He as carrier gas (7.4 mL min-1) and a GS-Q capillary column (30 m length, 0.530 UM ID) to separate the gases (Agilent 6890N) (Kracke et al. 2021). Pressure in serum bottles was measured with a pressure gauge (Ashcroft).

### Experimental Procedure

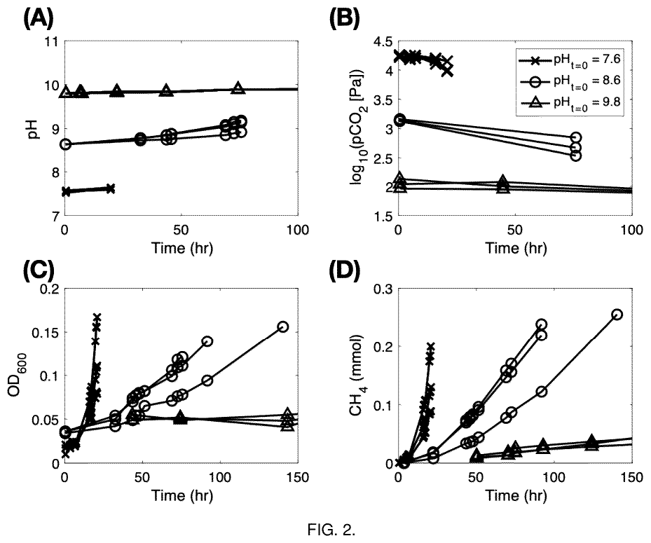

Growth observations in DIC-buffered medium. Growth of M. vannielii was characterized in mineral medium at pH 7.5-10. To prepare the serum bottles, 100 mM DIC was added to mineral medium from stock solutions. The DIC equilibrated with gaseous CO₂ overnight based on the pH of the solution, such that bottles at lower pH had more gaseous CO₂ and bottles at higher pH had more DIC at the start of the experiment (FIGS. 2, A and B), but they contained equal total moles of CO₂-species. To reduce lag times, bottles at each pH were inoculated from precultures growing within 0.5 pH units of the target pH. Of note, small pH variations between the target and actual pH can be attributed to temperature increase from 21° C. to 30° C. and to CO₂ evaporation into gas phase during equilibration of medium and stock solutions. A total of nine serum bottles were prepared with mineral medium and DIC buffer with three at pH 7.5, 8.5, and 10. After inoculation, OD, pH, pressure, CO₂ and CH₄ were measured over time until the cells grew above OD 0.10.

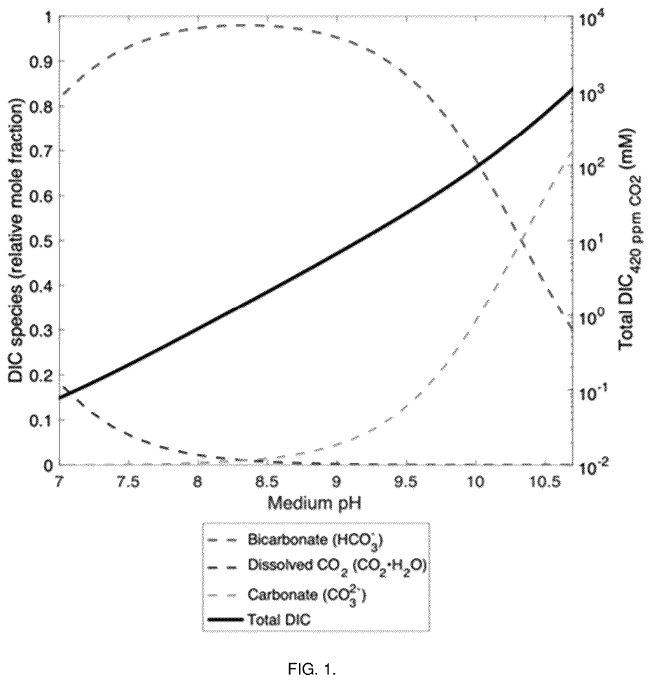

Specific methane production rates in non-DIC buffered medium. For examining the kinetics of CH₄ formation in M. vannielii as a function of pCO₂ and cDIC at pH 7, 8, and 9, seven serum bottles were prepared with CO₂-free pH buffered mineral medium, H₂ in excess, and 0-10 mM DIC. To prepare the medium, DIC stock solution was added to CO₂-free medium with 100 mM MOPS, 100 mM EPPS, or 15 mM Borate. After 20 minutes on a shaker at 30° C., the pCO₂ was stable. The cDIC in each bottle was calculated via carbon balance. At each pH, a linear pCO₂-DIC calibration curve was created from measurements of the seven serum bottles prior to inoculation (S. FIG. 1). Three additional sets of triplicate bottles with CO₂-free pH-buffered medium were amended with cDIC of 80 mM at pH 7, 60 mM at pH 8, and 100 mM at pH 9 to measure rates of methane formation with CO₂ in excess. In all experiments, the medium was sufficiently buffered such that pH was within 0.2 of the target pH after equilibration.

Next, 1 mL preculture grown in the same pH medium was transferred to each of the serum bottles. As a control for both CO₂ and CH₄ carryover, 1 mL preculture was filter-sterilized (0.2 μm) into a serum bottle without added DIC. The headspace of each serum bottle was first left to equilibrate for 20 min after inoculation, then measured at 30 min intervals for 4-8 hours. OD and pH were stable, and pressure dropped by less than 15% during the experiment (S. FIG. 2). CH₄ formation and pCO₂ were measured over time. cDIC during the experiment was calculated from the pCO₂-cDIC calibration curve at the appropriate pH. The experiment was repeated twice at each pH with similar results, though only one dataset was included here.

Continuous production of CH₄ from aerobic CO₂ source. Bio-CH₄ was produced from 5% CO₂-enriched air and H₂ from water electrolysis in a series of two trickle bed columns. To prepare the trickle bed columns, two glass columns (12″ long, 12 mm ID) were filled with cylindrical cores of polyurethane foam and plugged with rubber butyl stoppers at each end. The columns were sterilized then flushed with H₂. The first column was inoculated with 25 mL OD 0.15 pure culture of a halotolerant strain of Cupriavidus necator H16 (Adams et al. 2023). The second column was inoculated with 10 mL 0.3 OD pure culture of Methanococcus vannielii (DSM 1224). After inoculation, the columns were rolled horizontally for several hours on a shaker to allow cells to attach to the foam and initiate the biofilm. Then, columns were supplied with mineral medium in series at a dilution rate of 2 mL/hr, controlled by a peristaltic pump. Three 10 ml vials accumulated medium and gas for sampling before the first column, between the columns, and after the second column. Gas tight Viton tubing with Luer lock connections was used throughout the system. Columns were re-inoculated with M. vannielii and C. necator in the first few weeks of start up until CH₄ concentration increased in the outlet gas. While all parts were sterilized and treated with sterile protocols during start up of the columns, the columns were not treated with sterile methods in the weeks afterward.

The medium was prepared as described previously at pH 8 without added DIC. Immediately after removal from the autoclave, sterile DIC solution was added to reach 140 mM in the medium. Medium was then cooled and gas was supplied through a sterile filter, generating a headspace of simulated flue gas containing 76% N₂, 5% CO₂, and 19% O₂. The mixture was prepared in a 5 L gas bag using room air and gas tanks of N₂ and CO₂ (Linde, 4.5). The medium was stirred under the aerobic headspace to ensure equilibration throughout the experiment.

The co-culture was fed with H₂ from a lab-assembled electrolyzer. The electrolyzer consisted of a two-chambered borosilicate gastight H-cell (Adams & Chittenden, Scientific Glass, Berkeley, CA, USA). The two chambers each have a volume of 150 mL filled with 100 mL 100 mM NaSO₄, separated by a Nafion 117 proton-exchange membrane (Fuel Cell Store Inc., College Station, TX, USA, surface area 4.9 cm²). Both anode and cathode were platinized Ti mesh (TWL). A Potentiostat (VMP3; Bio-Logic Science Instruments, EC-Lab software version 11.21, Seyssinet-Pariset, France) maintained the current at 26 mA throughout the experiment. The produced H₂ was flowed over a Palladium catalyst in a rubber butyl stoppered glass test tube to remove trace O₂ that may have leaked across the PEM before the introduction of H₂ to the trickle bed columns.

The columns were regularly sampled to understand the flux of CO₂ and H₂ through the biological reactor. Gas samples (0.3 mL) were taken from the top of each column and from each of the sampling vials to measure pH₂, pCO₂, pCH₄, and pN₂ using a 0.5 mL glass syringe (Pressure-Lok, Vici Precision Sampling). Liquid samples (1 mL) were removed from the sampling vials to measure OD, pH, and dissolved inorganic carbon (DIC).

Gas was collected from the column in a 1 L glass bottle filled entirely with 2M NaCl solution and sealed with a rubber butyl stopper. The pressure from the feed and produced gas in the columns pushed NaCl solution into a connected glass bottle sealed with a rubber butyl stopper and filled with N₂ and connected to a gas bag, referred to as the ‘overflow.’ The columns and bottles were connected such that the final gas pressure in the outlet collection bottle was equal to atmospheric pressure. Gas and liquid flow rates were determined by the difference in the liquid levels between the outlet collection bottle and the overflow bottle once or twice a day. The gas flow rate was determined by the change in volume in the outlet bottle and the liquid flow rate was determined by the difference between the gas flow rate and the total liquid volume change between the two bottles. Volumes in each bottle were recorded once a day. The methane production rate at the time of sampling was 1.5 L L⁻¹ medium d⁻¹.

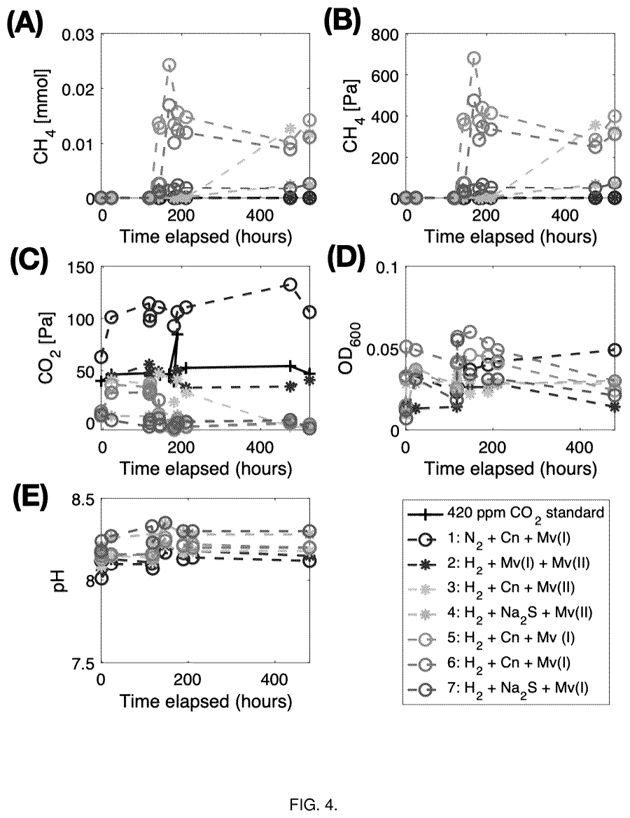

Oxygen removal from air-saturated alkaline medium. We demonstrated anaerobic metabolism from an oxygenated alkaline medium using both chemical and biological O₂ removal in a third experiment. In this experiment, mineral medium was prepared as described previously, at pH 8 with 100 mM EPPS and without added DIC. The sterile medium was equilibrated under room air at 23° C. on an incubation shaker. The medium was transferred in 25 mL aliquots to sealed 120 ml bottles that had been previously flushed with N₂ (bottle 1) or H₂ (bottle 2-7) and measured with gas chromatography to verify that each bottle contained very low amounts of CO₂ (<10 Pa). After medium was added to the bottles, bottles were shaken at 30° C. overnight. CO₂ concentrations in the gas phase were measured after equilibration at 30° C. to verify medium contained CO₂. To remove oxygen from the medium, knallgas bacterium, Cupriavidius necator, grown in equivalent medium that had been depleted in CO₂ (OD 0.4) was transferred to bottles 1, 3, 5, 6. DIC-free 2 mM Na₂S was added to bottles 4 and 7. Bottles were shaken at 30° C. for a few days. Once oxygen was removed, M. vannielii grown in medium that had been previously reduced by C. necator (OD 0.3) was added to each bottle 1, 5, 6, and 7. An equal amount of grown M. vannielii was added to bottle 2 to demonstrate the requirement of biologically compatible oxygen removal for anaerobic metabolism. As expected, only the bottles where oxygen was removed either chemically or biologically produced CH₄ and removed CO₂, specifically bottles 5, 6, and 7. Once CH₄ production stopped, 1 mL inoculum from bottle 5, 6, and 7 was transferred to bottle 2, 3, and 4, respectively (FIG. 4, A-E).

### Results and Discussion

Growth of Methanococcus vannielii with low pCO₂. To explore the capacity of M. vannielii to convert atmospheric CO₂ levels (42 Pa, equivalent to cDIC of approximately 0.1-10 mM depending on pH, to CH₄), two experiments were conducted. First, batch growth experiments were conducted across a pH range of 7.5-10. During growth, pH increased in all cultures due to overall CO₂ consumption in methanogenesis (FIG. 2, A). The observed doubling times at pHt=0 7.6, 8.6, and 9.8 were 6.5 hr, 12 hr, and 22 hr, respectively and methane formation rates followed a similar trend (FIGS. 2, C and D). Still, it was unclear whether the rates of growth and methane formation decreased in M. vannielii because of physiological constraints imposed by pH or because of low concentration of available CO₂ (gas) as substrate (FIG. 2, B).

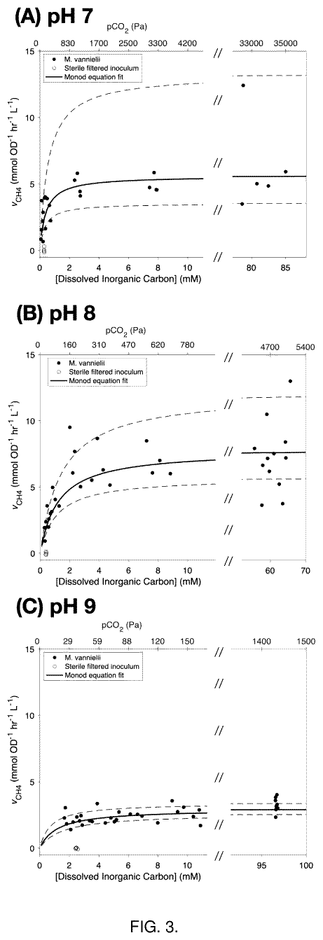

Specific rates of methane formation at low pCO₂ and cDIC at pH 7, 8, and 9. To determine the apparent kinetic parameters, K_{M} and Vmax, of M. vannielii for CO₂ consumption, the initial rates of methane formation from a range of pCO₂ and cDIC were determined at pH 7, 8, and 9 in well-buffered medium. Lineweaver-Burk plotting was used to determine apparent K_{M} and vmax values at each pH (FIG. 3). CH₄ formation rates were highest at 2-10 mM DIC at pH 8. At pH 7, 8, and 9, the vmax was calculated from the fitted curves to be 5.4 mmol CH₄ L⁻¹ OD⁻¹ hr 1, 9.7 mmol CH₄ L⁻¹ OD⁻¹ hr⁻¹, and 2.6 mmol CH₄ L⁻¹ OD⁻¹ hr⁻¹, respectively. At pH 7, 8, and 9, the apparent KM values for cDIC were 0.4 mM, 1.6 mM, and 0.8 mM and for pCO₂ were 160 Pa, 135 Pa, and 10 Pa, respectively (Table 1). While the methane formation rates follow a clear Monod trend at pH 7 and 8, the methane formation rates at pH 9 were relatively constant across the cDIC range of 2 mM to 100 mM (FIG. 3, C). The apparent K_{M} value at pH 9 was most likely below 2 mM DIC or 25 Pa CO₂. Still, the calculated K_{M} defined here was extrapolated and should be treated with caution because quantification of the very low pCO₂ (<10 Pa) was unreliable. The finding of higher specific methane production rates at pH 8 and the decreasing growth rates with increasing pH indicates that the optimal pH for growth of M. vannielii in the described medium is likely between pH 7.5-8 (Stadtman and Barker, 1951).

Most notably, the rate of CH₄ formation at the atmospheric pCO₂ of 42 Pa, vₐₜₘ, was 1.1 mmol CH₄ L⁻¹ OD⁻¹ hr⁻¹, 2.4 mmol CH₄ L⁻¹ OD⁻¹ hr⁻¹, and 2.1 mmol CH₄ L⁻¹ OD⁻¹ hr⁻¹ at pH 7, 8, and 9, respectively (FIG. 3, Table 1). At all pH values, the threshold CO₂ concentration below which methane formation was no longer detected, Sₘᵢₙ, was below atmospheric concentrations and below the reliable measurement range, at about 8 Pa, 6 Pa, and 7 Pa at pH 7, 8, and 9, respectively. Therefore, M. vannielii can convert more than 75% of atmospheric pCO₂ to CH₄ at biologically significant rates.

TABLE 1
Apparent values for K_{M}, vₘₐₓ, vₐₜₘ, and Sₘᵢₙ of M. vannielii.

| Parameters | | pH 7 | | pH 8 | | pH 9 | |
|---|---|---|---|---|---|---|---|
| K_{M} (pCO₂ ) | Pa | 168 | [149, 240] | 100 | [90, 117] | 18* | [11, 22] |
| K_{M} (cDIC) | mM | 0.4 | [0.35, 0.57] | 1.2 | [1.1, 4.4] | 1.2* | [0.8, 1.6] |
| vₘₐₓ | mmol CH₄ L⁻¹ OD⁻¹ hr⁻¹ | 5.6 | [3.5, 13.2] | 7.8 | [5.7, 12.1] | 2.9 | [2.6, 3.4] |
| vₐₜₘ (42 Pa) | mmol CH₄ L⁻¹ OD⁻¹ hr⁻¹ | 1.1 | [0.8, 2.0] | 2.3 | [1.8, 3.2] | 2.1 | [1.7, 2.7] |
| Sₘᵢₙ (pCO₂ ) | Pa | 8 | [6, 10] | 6 | [5, 7] | 7 | [4, 13] |
| Sₘᵢₙ (cDIC) | mM | 0.02 | [0.01, 0.03] | 0.07 | [0.06, 0.08] | 0.5 | [0.4, 0.6] |

*indicates an extrapolated value. [ ] indicates 95% confidence interval.
Table 1: Apparent KM, vmax, vatm, and Smin determined from methane production rates of M. vannielii in buffered mineral medium. See Callander, Grace, Jörg S. Deutzmann, and Alfred M. Spormann. “Alkaline hydrogenotrophic methanogenesis in Methanococcus vannielii at low carbon dioxide concentrations.” Journal of CO2 Utilization 83 (2024): 102788.

Feasibility of using M. vannielii to capture and convert dilute CO₂. To our knowledge, the rate of methane production under CO₂ limited conditions has only been reported in Methanobacterium congolense, a bacterium found in anaerobic digesters. At a pH range of 6.5-7, the apparent K_{M} for M. congolense was 2.2 mM, similar to the apparent K_{M} values for cDIC documented in this study. Chen et al. hypothesized via modeling of the pCO₂-CDIC equilibration that methanogenesis rates in M. congolense would decrease with increasing pH due to reduced pCO₂. In contrast, the methane production rates in our study show comparable rates among the tested pH's of 7, 8, and 9 from 0-5 mM DIC, even though pCO₂ decreased by three orders of magnitude across this pH and cDIC range (FIG. 3, A-C). In other words, the apparent K_{M} values in M. vannielii for cDIC are similar across all pH's tested, while the apparent K_{M} values for pCO₂ decrease with increasing pH. Moreover, while CO₂ and DIC are in equilibrium, the results here support the hypothesis that concentration of bicarbonate rather than pCO₂ determines the rate of methane formation in M. vannielii. It is feasible to concentrate atmospheric concentrations of CO₂ as DIC in pH 8 or 9 solution and convert it in aqueous solution to CH₄ at 30% Vmax, or 2.4 mmol CH₄ L⁻¹ OD 1 hr-1.

To convert atmospheric CO₂ directly using anaerobic microbial metabolism, an additional process must remove O₂ from the system. Methanogenesis enzymes and cofactors, such as methyl-coenzyme M reductase or ferredoxin, are inactivated by molecular oxygen, which is present in atmospheric and most industrial CO₂ waste streams. M. vannielii is an especially oxygen sensitive strain, but some methanogens do recover after oxygen exposure. Instead of flushing with O₂-free gas or boiling the medium, O₂ removal without CO₂ removal from solution can utilize either chemical or biological processes. Chemical processes to remove oxygen include, but are not limited to, the addition of reduced sulfur compounds such as Na₂S or Na₂S₂O₄. Biological processes to remove O₂ include, but are not limited to, addition of aerobic microbes that consume oxygen through their metabolism. A particularly interesting metabolism is that of autotrophic knallgas bacteria, which consume H₂ and O₂ to produce water.

We demonstrated that anaerobic metabolism can reduce trace CO₂ to CH₄ using chemical and biological means for O₂ removal in batch and continuous processes. In the batch experiment, as expected, only the bottles where oxygen was removed either chemically, using Na₂S, or biologically, using a co-culture with C. necator, produced CH₄ and removed CO₂. The CH₄ produced in this experiment was 0.1-0.3% of the total gas volume and CO₂ was removed below the detection limit (<10 Pa). In the continuous experiment, 5% CO₂-enriched air was converted to 20-30% CH₄ in H₂ in a trickle bed reactor inoculated with both C. necator and M. vannielii. The unoptimized reactor produced CH₄ continuously for two weeks with an average CO₂ conversion efficiency to CH₄ of 50%. Further experiments may be designed such that DIC and H₂ are consumed completely at high product yield and the produced CH₄ is a relatively high purity, self-separating, reduced carbon compound.

Bicarbonate versus CO₂ utilization in M. vannielii at alkaline pH. The correlation between methane formation rates and cDIC raises questions about the biologically preferred form of CO₂ for uptake in methanogenesis. CO₂ diffuses across the cytoplasmic membrane into the cells and no active CO₂ transport mechanism is known. On the other hand, uptake of bicarbonate requires a mechanism for import, and a bicarbonate transport system has not been reported in methanogens to our knowledge. We conducted a BLAST analysis of the genome of a strain of M. vannielii (not used in this study) and found the genome to encode a protein similar to a low-affinity, high-flux, Na⁺-dependent active HCO₃⁻ transporter (WP_012066491.1, E=3×10⁻⁵⁴). If this or a similar transporter is involved in bicarbonate uptake during methanogenesis in M. vannielii, the coupled Na⁺-HCO₃⁻ transport will require metabolic energy and reduce ATP yield as energy conservation is driven by a transmembrane Na+ gradient. Import of bicarbonate as substrate for methanogenesis could also be coupled to carbonic anhydrase activity. The strain used in this study, M. vannielii SB, is predicted to encode proteins similar to a characterized β-class (ABR54117.1, E=10⁻³⁹) and a γ-class (ABR54964.1, E=10⁻⁴⁹) carbonic anhydrase.

Scalability of hydrogenotrophic methanogenesis for dilute CO₂ conversion. Designing around the thermodynamic penalty of atmospheric CO₂ removal is key to its efficacy for global warming mitigation. The thermodynamic minimum work required to concentrate CO₂ from 0.04% to 100% is 450 MJ/ton CO₂. In reality, 4-5 times more energy is required due to heat and mass transfer limitations, poor reaction kinetics, and solvent or sorbent regeneration via pressure, temperature, or electrochemical pH swing processes. Reactive capture with M. vannielii would bypass the separation and sorbent regeneration steps by converting CO₂ from ambiently preloaded alkaline solutions with selective, self-replicating organisms. The produced methane is a low solubility gas, and, therefore, additional infrastructure is not required for methane purification.

While methanogenesis simplifies the process of dilute CO₂ capture, CO₂ reduction to CH₄ requires H₂, which consumes >5 GJ/ton CO₂, depending on the H₂ source. Therefore, once CH₄ is produced, it would improve the efficiency of the system to further react the CH₄ to form stable products that facilitate energy recovery, such as high-quality carbon materials and hydrogen via methane pyrolysis. Moreover, microbial methanogenesis is a nature-based dilute CO₂ capture and conversion technology that is simultaneously compatible with existing energy infrastructure and quantifiably produces microbially inaccessible carbon for storage over long time periods.

Microbial methanogenesis has the potential to convert CO₂ at atmospheric concentration or ambiently enriched DIC from alkaline solution at ambient temperatures and pressures. Rates of methane production measured here for M. vannielii under atmospheric pCO₂ and cDIC were highest at pH 8:2.4 mmol CH₄ L⁻¹ OD⁻¹ hr 1 or 30% Vmax.

### REFERENCES

Adli, N. M., Zhang, H., Mukherjee, S., Wu, G., 2018. Review—Ammonia Oxidation Electrocatalysis for Hydrogen Generation and Fuel Cells J. Electrochem. Soc. 165 J3130.
Berg, I., Kockelkorn, D., Ramos-Vera, W., Say, R., Zarzycki, J., Hügler, M., Alber, B., Fuchs, G., 2010. Autotrophic carbon fixation in archaea. Nature Reviews Microbiology, 447-460, 8(6). DOI: 10.1038/nrmicro2365
Buesseler, K., Jin, D., Kourantidou, M., Levin, D., Ramakrishna, K., Renaud, P., 2022. The Ocean Twilight Zone's Role in Climate Change. Woods Hole Oceanographic Institution. DOI: 10.1575/1912/28074
Castro-Muñoz, R., Ahmad, M. Z., Malankowska, M., Coronas, J., 2022. A new relevant membrane application: CO₂ direct air capture (DAC). Chemical Engineering Journal, 446, 2. DOI: 10.1016/j.cej.2022.137047
Chen, X., Ottosen, L., Kofoed, M., 2019. How low can you go: Methane production of methanobacterium congolense at low CO₂ concentrations. Frontiers in Bioengineering and Biotechnology, 7(MAR) DOI: 10.3389/fbioe.2019.00034
Chi, Z., Xie, Y., Elloy, F., Zheng, Y., Hu, Y., Chen, S, 2013. Bicarbonate-based Integrated Carbon Capture and Algae Production System with alkalihalophilic cyanobacterium. Bioresource Technology, (2013), 513-521, 133. DOI: 10.1016/j.biortech.2013.01.150
Crooks and Donnellan 1989. Kinetics and Mechanism of the Reaction between Carbon Dioxide and Amines in Aqueous Solution. J. Chem. Soc. Perkin Trans., 331-333, II. 8/01668B
Deutzmann, J. S., Callander, G., Gu, W., Müller, A. L., McCully, A. L., Ahn, J. K., Kracke, F. and Spormann, A. M., 2022. Low-Cost Clamp-On Photometers (ClampOD) and Tube Photometers (TubeOD) for Online Cell Density Determination. Frontiers in Microbiology, 12, p. 790576.
Freyman, M., Huang, Z., Ravikumar, D., Duoss, E., Li, Y., Baker, S., Pang, S., Schaidle, J., 2023. Reactive CO₂ capture: A path forward for process integration in carbon management. Joule, 631-651, 7 (4). DOI: 10.1016/J.JOULE.2023.03.013
Gasser, T., Guivarch, C., Tachiiri, K., Jones, C., Ciais, P., 2015. Negative emissions physically needed to keep global warming below 2° C. Nat. Commun., 8958, 6. DOI: 10.1038/ncomms8958
Harrison, K., Dowe, N., 2021. Collaboration to Enable Higher Penetrations of Solar Power Generation Using the Natural Gas Pipeline System for Energy Storage: Cooperative Research and Development Final Report, CRADA Number CRD-14-00567.
Jarrell, K., 1985. Extreme Oxygen Sensitivity in Methanogenic Archaebacteria. BioScience, Vol. 35, No. 5, pp. 298-302.
Jeyakanthan, J., Rangarajan, S., Mridula, P., Kanaujia, S. P., Shiro, Y., Kuramitsu, S., Yokoyama, S. and Sekar, K., 2008. Observation of a calcium-binding site in the γ-class carbonic anhydrase from Pyrococcus horikoshii. Acta Crystallographica Section D: Biological Crystallography, 64 (10), pp. 1012-1019.
Jones, J. B., Stadtman, T. C., 1981. Selenium-dependent and selenium-independent formate dehydrogenases of Methanococcus vannielii. Separation of the two forms and characterization of the purified selenium-independent form. Journal of Biological Chemistry, 256, 2, 656-665.
Kim, S., Nitzsche, M. P., Rufer, S. B., Lake, J. R., Varanasi, K. K., Hatton, T. A., 2023. Asymmetric chloride-mediated electrochemical process for CO₂ removal from oceanwater. Energy & Environmental Science, 16, 2030-2044. DOI: 10.1039/D2EE03804H
Keith, D., Holmes, G., St. Angelo, D., Heidel, K., 2018. A Process for Capturing CO₂ from the Atmosphere. Joule, 1573-1594, 2 (8). DOI: 10.1016/j.joule.2018.05.006
Kracke, F., Deutzmann, J. S., Gu, W., Spormann, A. M., 2020. In situ electrochemical H₂ production for efficient and stable power-to-gas electromethanogenesis. Green Chemistry, 22, 6194. DOI: 10.1039/d0gc01894e
Kracke, F., Deutzmann, J. S., Jayathilake, B. S., Pang, S. H., Chandrasekaran, S., Baker, S. E., Spormann, A. M., 2021. Efficient Hydrogen Delivery for Microbial Electrosynthesis via 3D-Printed Cathodes. Frontiers in Microbiology, 12.
Kozono, D., Ding, X., Iwasaki, I., Meng, X., Kamagata, Y., Agre, P., Kitagawa, Y., 2003. Functional Expression and Characterization of an Archaeal Aquaporin. Journal of Biological Chemistry, 278, 12, 10649-10656.
Lan, X., Tans, P., 2023. Trends in globally-averaged CO₂ determined from NOAA Global Monitoring Laboratory measurements. Accessed Aug. 21, 2023.
Lineweaver, H., Burk, D., 1934. The Determination of Enzyme Dissociation Constants. Journal of the American Chemical Society, 658-666, 56 (3). DOI: 10.1021/JA01318A036/ASSET/JA01318A036.FP.PNG_V03
Mayer, F., Enzmann, F., Lopez, A., Holtmann, D., 2019. Performance of different methanogenic species for the microbial electrosynthesis of methane from carbon dioxide. Bioresource Technology, 289. DOI: 10.1016/j.biortech.2019.121706
McQueen, N., Gomes, K., McCormick, C., Blumanthal, K., Pisciotta, M., Wilcox, J., 2021. A review of direct air capture (DAC): Scaling up commercial technologies and innovating for the future. Progress in Energy, 3 (3). DOI: 10.1088/2516-1083/abf1ce
Millero, F., 1979. The thermodynamics of the carbonate system in seawater. Geochimica et Cosmochimica Acta, 1651-1661, 43.
Mitchell, M., Jensen, O., Cliffe, K., Maroto-Valer, M., 2010. A model of carbon dioxide dissolution and mineral carbonation kinetics. Proceedings of the Royal Society A: Mathematical, Physical and Engineering Sciences, 1265-1290, 466 (2117).
Monod, J., 1949. The growth of bacterial cultures. Annu. Rev. Microbiol. 3:371-394.
Pierrot, D., Epitalon, J.-M., Orr, J.C., Lewis, E., Wallace, D. W. R., MS Excel program developed for CO₂ system calculations-version 2.1, (2012), GitHub repository,
Price, G., Woodger, F., Badger, M., Howitt, S., Tucker, L. Identification of a SulP-type bicarbonate transporter in marine cyanobacteria. Proc Natl Acad Sci USA. 2004 Dec. 28; 101 (52): 18228-33. DOI: 10.1073/pnas.0405211101.
Realmonte, G., Drouet, L., Gambhir, A., Glynn, J., Hawkes, A., Köberle, A. C., and Tavoni, M., 2019. An inter-model assessment of the role of direct air capture in deep mitigation pathways. Nat Commun 10, 3277.
Sánchez-Bastardo, N., Schlögl, R., Ruland, H., 2021. Methane Pyrolysis for Zero-Emission Hydrogen Production: A Potential Bridge Technology from Fossil Fuels to a Renewable and Sustainable Hydrogen Economy. Industrial & Engineering Chemistry Research. 60 (32), 11855-11881. DOI: 10.1021/acs.iecr. 1c01679
Schneider, S., Bajohr, S.; Graf, F.; Kolb, T., 2020. State of the art of hydrogen production via pyrolysis of natural gas. ChemBioEng Rev., 7 (5), 150-158. DOI: 10.1002/cben.202000014
Smith, K., Ferry, J., 2000. Prokaryotic carbonic anhydrases. FEMS Microbiology Reviews, (2000), 335-366, 24 (4), DOI: 10.1111/j.1574-6976.2000.tb00546.x
Stadtman, T., Barker, H., 1951. Studies on the methane fermentation x. A new formate-decomposing bacterium, Methanococcus vannielii. Journal of Bacteriology, 62, 3. DOI: 10.1128/jb.62.3.269-280.1951
Strefler, J., Bauer, N., Elmar, K., Popp, A., Giannousakis, A., Edenhofer, O., 2018. Between Scylla and Charybdis: Delayed mitigation narrows the passage between large-scale CDR and high costs. Environmental Research Letters, 13. DOI: 10.1088/1748-9326/11b2ba
Strop, P., Smith, K., Iverson, T., Ferry, J., Rees, D., 2001. Crystal Structure of the “cab”-type B Class Carbonic Anhydrase from the Archaeon Methanobacterium thermoautotrophicum. Enzyme catalysis and regulation, 276, 13. DOI: 10.1074/jbc.M009182200
Sun, E., Zhai, S., Kim, D., Gigantino, M., Haribal, V., Dewey, O. S., Williams, S. M., Wan, G., Nelson, A., Marin-Quiros, S., Martis, J., Zhou, C., Oh, J., Randall, R., Kessler, M., Kong, D., Rojas, J., Tong, A., Xu, X., Huff, C., Pasquali, M., Gupta, R., Cargnello, M., Majumdar, A., 2023. A semi-continuous process for co-production of CO₂-free hydrogen and carbon nanotubes via methane pyrolysis. Cell Reports Physical Science, 4, 101338. DOI: 10.1016/j.xcrp.2023.101338
Thauer, R., Kaster, A., Seedorf, H., Buckel, W., Hedderich, R., 2008. Methanogenic archaea: Ecologically relevant differences in energy conservation. Nature Reviews Microbiology, 579-591, 6 (8). DOI: 10.1038/nrmicro1931
Vorholt, J., Thauer, R., 1997. The active species of ‘CO₂’ utilized by formylmethanofuran dehydrogenase from methanogenic Archaea. European Journal of Biochemistry, 919-924, 248 (3). DOI: 10.1111/j.1432-1033.1997.00919.x
Wang, C., Sun, B., Zhang, X., Huang, X., Zhang, M., Guo, H., Chen, X., Huang, F., Chen, T., Mi, H., Yu, F., Liu, L., Zhang, P., 2019. Structural mechanism of the active bicarbonate transporter from cyanobacteria. Nature Plants, 1184-1193, 5 (11). DOI: 10.1038/s41477-019-0538-1
Welch, A., Dunn, E., Duchene, J., Atwater, H., 2020. Bicarbonate or Carbonate Processes for Coupling Carbon Dioxide Capture and Electrochemical Conversion. ACS Energy Letters, (2020), 940-945, 5 (3). DOI: 10.1021/acsenergylett.0c00234
Wilcox, J., 2012. Carbon capture. Springer Science & Business Media.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

What is claimed is:

1. A method for the microbial conversion of dissolved inorganic carbon (DIC) in an anoxic alkaline solution to a reduced carbon compound, the method comprising:
contacting the anoxic alkaline solution with an effective dose of an alkalotolerant or alkaliphilic microbe; and
maintaining for a period of time sufficient to convert the DIC to a reduced carbon compound.

2. The method of claim 1, wherein the microbe is a hydrogenotrophic methanogen.

3. The method of claim 1, wherein the reduced carbon compound is methane.

4. The method of claim 1, wherein the alkaline solution is treated to decrease alkalinity.

5. The method of claim 1, wherein the solution is from pH 7 to pH 11 at the time of contacting with the microbe.

6. The method of claim 1, wherein the concentration of DIC is from about 100 μM to 10 mM, or from about 10 μM to 10 mM, or from about 1 mM to 300 mM.

7. The method of claim 1 wherein anoxic conditions are chemically or biologically created.

8. The method of claim 1 wherein anoxic conditions are created by separating oxygen from the alkaline solution by a DIC selective membrane, by consuming oxygen via a chemical reaction, or by co-cultivation with an oxygen-consuming microorganism, optionally prior to or concurrent with addition of an anaerobic microbe capable of catabolic CO₂ reduction.

9. The method of claim 1, wherein the alkaline solution comprises less than about 500 μg O₂ L⁻¹.

10. The method of claim 1, wherein the alkalotolerant or alkaliphilic microbe is one or more members of the Methanopyrales, Methanococcales, Methanobacteriales, Methanomicrobiales, Methanocellales, and Methanosarcinales, optionally Methanococcus vannielii.

11. The method of claim 1 wherein the alkalotolerant or alkaliphilic microbe is a member of a complex or defined microbial community.

12. The method of claim 1, wherein the reduced carbon compound, optionally methane, is removed from the system and optionally converted to a longer chain hydrocarbon or carbon product.

13. The method of claim 1 wherein the alkaline solution is sea water.

14. The method of claim 1, wherein the process is performed in an anaerobic bioreactor.

15. The method of claim 14, wherein the bioreactor comprises a component for removal of excess O₂.

16. The method of claim 14, wherein the bioreactor comprises an input for the alkaline solution.

17. The method of claim 14, wherein the bioreactor is supplied with a continuous inflow of solution.

18. The method of claim 14, wherein the bioreactor solution is maintained without exchange for an extended period of time.

19. The method of claim 14, wherein the bioreactor comprises a methane storage unit operably connected to the bioreactor and capable of storing methane produced by the bioreactor.

20. A system for use in the method of claim 1.
